(19) United States
(12) Patent Application Publication
(10) Pub. No.: US 2025/0134423 A1
(43) Pub. Date: May 1, 2025

(54) HYPERSATURATION INDEX

(71) Applicant: Masimo Corporation, Irvine, CA (US)

(72) Inventors: Mohamed K. Diab, Ladera Ranch, CA (US); Prashanth Iyengar, Irvine, CA (US); Anand Sampath, Irvine, CA (US); Ammar Al-Ali, San Juan Capistrano, CA (US)

(21) Appl. No.: 18/944,506

(22) Filed: Nov. 12, 2024

Related U.S. Application Data
(63) Continuation of application No. 17/352,805, filed on Jun. 21, 2021, now Pat. No. 12,167,913.
Application No. 17/352,805 is a continuation of application No. 16/881,930, filed on May 22, 2020, now Pat. No. 11,071,480.
Application No. 16/881,930 is a continuation of application No. 16/674,812, filed on Nov. 5, 2019, now Pat. No. 10,674,948.
Application No. 16/674,812 is a continuation of application No. 15/705,761, filed on Sep. 15, 2017, now Pat. No. 10,531,819.
Application No. 15/705,761 is a continuation of application No. 14/852,356, filed on Sep. 11, 2015, now Pat. No. 9,775,546.
Application No. 14/852,356 is a continuation of application No. 13/865,081, filed on Apr. 17, 2013, now Pat. No. 9,131,881.
(60) Provisional application No. 61/625,599, filed on Apr. 17, 2012.
(60) Provisional application No. 61/703,087, filed on Sep. 19, 2012.
(60) Provisional application No. 61/719,866, filed on Oct. 29, 2012.

(51) Int. Cl. A61B 5/1455; A61B 5/00; A61B 5/0205

(52) U.S. Cl. CPC A61B 5/14552; A61B 5/0205; A61B 5/14551; A61B 5/7282; A61B 5/7405; A61B 5/743; A61B 5/746; A61B 5/6826; A61B 2562/0238; A61M 2230/205

(57) ABSTRACT

Embodiments of the present disclosure provide a hypersaturation index for measuring a patient's absorption of oxygen in the blood stream after a patient has reached 100% oxygen saturation. This hypersaturation index provides an indication of the partial pressure of oxygen of a patient. In an embodiment of the present invention, a hypersaturation index is calculated based on the absorption ratio of two different wavelengths of energy at a measuring site. In an embodiment of the invention, a maximum hypersaturation index threshold is determined such that an alarm is triggered when the hypersaturation index reaches or exceeds the threshold. In another embodiment, an alarm is triggered when the hypersaturation index reaches or falls below its starting point when it was first calculated.

### PRIORITY CLAIM TO RELATED PROVISIONAL APPLICATIONS

The present application is a continuation of U.S. application Ser. No. 17/352,805, filed Jun. 21, 2021 entitled “Hypersaturation Index,” which is a continuation of U.S. application Ser. No. 16/881,930, filed May 22, 2020 entitled “Hypersaturation Index,” now U.S. Pat. No. 11,071,480, which is a continuation of U.S. application Ser. No. 16/674,812, filed Nov. 5, 2019 entitled “Hypersaturation Index,” now U.S. Pat. No. 10,674,948, which is a continuation of U.S. application Ser. No. 15/705,761, filed Sep. 15, 2017, entitled “Hypersaturation Index,” now U.S. Pat. No. 10,531,819, which is a continuation of U.S. application Ser. No. 14/852,356, filed Sep. 11, 2015, entitled “Hypersaturation Index,” now U.S. Pat. No. 9,775,546, which is a continuation of U.S. application Ser. No. 13/865,081, filed Apr. 17, 2013, entitled “Hypersaturation Index,” now U.S. Pat. No. 9,131,881, which claims priority benefit under 35 U.S.C. § 119 (c) of U.S. Provisional Application Ser. No. 61/719,866, filed Oct. 29, 2012, entitled “Noninvasive Partial Pressure of Oxygen Sensing System,” U.S. Provisional Application Ser. No. 61/703,087, filed Sep. 19, 2012, entitled “Noninvasive Partial Pressure of Oxygen Sensing System,” and U.S. Provisional Application Ser. No. 61/625,599, filed Apr. 17, 2012, entitled “Noninvasive Partial Pressure of Oxygen Sensing System,” the disclosures of which are incorporated herein by reference.

### FIELD OF THE INVENTION

The present invention relates to the field of noninvasive oxygen delivery measurement using optical based sensors.

### BACKGROUND

The measurement of oxygen delivery to the body and the corresponding oxygen consumption by its organs and tissues is vitally important to medical practitioners in the diagnosis and treatment of various medical conditions. Oxygen delivery is useful, for example, during certain medical procedures, where artificially providing additional oxygen to the patient's blood stream may become necessary. For example, during an intubation procedure, the patient will stop breathing while the procedure is performed. The patient is typically provided with oxygen before the intubation procedure. Because the patient stops breathing during an intubation procedure, the patient's blood oxygen saturation level will fall. In that situation, the medical practitioner must ensure that the patient has sufficient reserves of oxygen in their system before intubation so that during the intubation procedure suffocation is avoided. At the same time, providing oxygen at a high pressure to a patient can cause damage to the alveoli of an adult patient. On the other hand, even normal oxygen levels can or cause blindness in neonatal patients.

The current standard of care is to measure oxygen delivery through the use of a pulse oximeter. Pulse oximeters measure oxygen saturation (SpO₂). SpO₂ represents the percent of available hemoglobin that can chemically bind with oxygen molecules.

Another indicator of oxygen delivery is the partial pressure of oxygen (PaO₂), However, there are currently no reliable ways to measure PaO₂ noninvasively. Invasive PaO₂ measurements require expensive sensors and are known to carry serious side effects that can harm the health of a patient.

### SUMMARY

Embodiments of the present disclosure provide a hypersaturation index for measuring a patient's absorption of oxygen in the blood stream after a patient has reached 100% oxygen saturation. This hypersaturation index provides an indication of an increased level of dissolved oxygen in the plasma. This is useful, for example, for patients that are on supplemental oxygen therapy or are on a ventilator or closed-loop positive pressure delivery device. An excessively high level of PaO₂ can be dangerous for most patients. In some patients, for example neonates, a high level of PaO₂ can cause loss of eyesight. Significant damage can occur to the lungs, and in particular, to the alveoli structures in the lungs, if the PaO₂ level is too high.

In another embodiment, a timer is provided that indicates when a hypersaturated patient is likely to return to a baseline saturation level after oxygen administration is stopped. This is useful, for example, during an intubation procedure.

Pulse oximetry is a noninvasive technique which allows the continuous in vivo measurement of arterial oxygen saturation and pulse rate in conjunction with generation of a photoplethsymograph waveform. Measurements rely on sensors which are typically placed on the fingertip of an adult or the foot of an infant. As explained in detail below, the ratio of red and infrared light signals absorbed at the measuring site is calculated (R/IR ratio). Oxygen saturation level is determined using a lookup table that is based on empirical formulas that convert the ratio of red and infrared absorption rates to a SpO₂ value.

A correlation exists between the R/IR ratio and the level of PaO₂. This relationship between R/IR ratio and PaO₂ levels, however, varies from patient to patient. For example, at the same PaO₂ level, one patient may have a R/IR ratio of 0.55 and another patient may have a reading of 0.45. Therefore, once the absorption level reaches 100%, it becomes difficult for the medical practitioner to assess the patient's condition with respect to PaO₂ and the potential dangers of a high level of PaO₂. Without the ability to accurately measure the PaO₂ level, medical practitioners are in need of a noninvasive way to monitor a patient's hypersaturation status.

In an embodiment of the present invention, a hypersaturation index is calculated based on the reading of the R/IR ratio at the measurement site. In an embodiment of the invention, a maximum hypersaturation index threshold is determined such that an alarm is triggered when the hypersaturation index reaches or exceeds the threshold. In another embodiment, an alarm is triggered when the hypersaturation index reaches or falls below its starting point when it was first calculated.

### BRIEF DESCRIPTION OF THE DRAWINGS

The drawings and following associated descriptions are provided to illustrate embodiments of the present disclosure and do not limit the scope of the claims. Corresponding numerals indicate corresponding parts, and the leading digit of each numbered item indicates the first figure in which an item is found.

FIG. 1 illustrates a perspective view of a patient monitoring system in accordance with an embodiment of the disclosure.

FIG. 2 illustrates a block drawing of a patient monitoring system in accordance with an embodiment of the disclosure.

FIG. 3A-3B illustrate graphs of SpO₂ versus PaO₂.

FIG. 3C illustrates a saturation calibration curve.

FIG. 3D-3E illustrate graphs of the difference between arterial and venous saturation vs. perfusion index.

FIG. 3F illustrates the graph of the ratio of R/IR and PaO₂ vs. time.

FIG. 4 illustrates a graph of SpO₂ versus the R/IR ratio and a hypersaturation index versus the R/IR ratio.

FIG. 5 illustrates a flowchart depicting an embodiment of the invention.

FIG. 6 illustrates a flowchart depicting an embodiment of the invention.

FIG. 7 illustrates a visualization of an indication of hypersaturation according to an embodiment of the invention.

FIG. 8A-8B illustrate visualizations of indications of hypersaturation according to an embodiment of the invention.

FIGS. 9A and 9B illustrate a timer display illustrating when a hypersaturated patient will return to a normal saturation level.

FIGS. 10A and 10B illustrate an alternative embodiment of a timer display.

FIG. 11 illustrates another alternative embodiment of a timer display.

### DETAILED DESCRIPTION

Aspects of the disclosure will now be set forth in detail with respect to the figures and various embodiments. One of skill in the art will appreciate, however, that other embodiments and configurations of the devices and methods disclosed herein will still fall within the scope of this disclosure even if not described in the same detail as some other embodiments. Aspects of various embodiments discussed do not limit the scope of the disclosure herein, which is instead defined by the claims following this description.

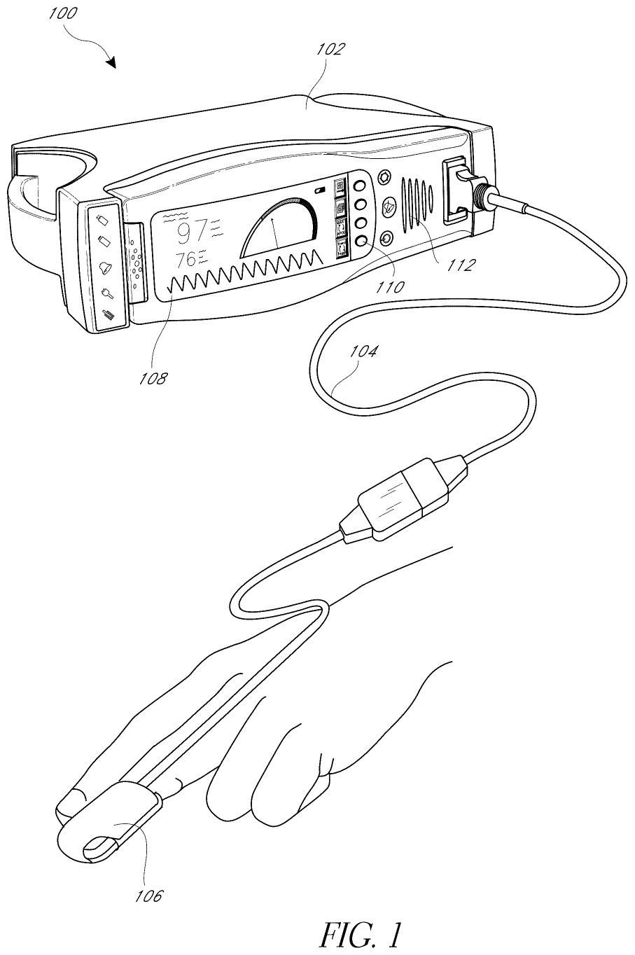

Turning to FIG. 1, a patient monitoring system 100 is illustrated. The patient monitoring system 100 includes a patient monitor 102 attached to a sensor 106 by a cable 104. The sensor monitors various physiological data of a patient and sends signals indicative of the parameters to the patient monitor 102 for processing. The patient monitor 102 generally includes a display 108, control buttons 110, and a speaker 112 for audible alerts. The display 108 is capable of displaying readings of various monitored patient parameters, which may include numerical readouts, graphical readouts, and the like. Display 108 may be a liquid crystal display (LCD), a cathode ray tube (CRT), a plasma screen, a Light Emitting Diode (LED) screen, Organic Light Emitting Diode (OLED) screen, or any other suitable display. A patient monitoring system 102 may monitor oxygen saturation (SpO2), hypersaturation, perfusion index (PI), pulse rate (PR), hemoglobin count, and/or other parameters.

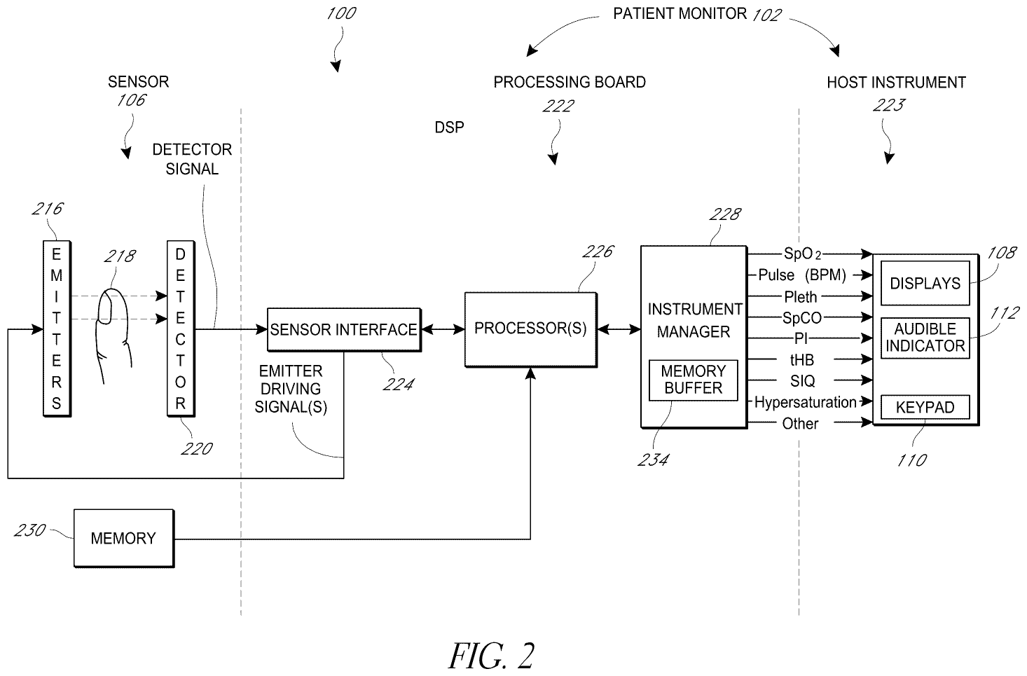

FIG. 2 illustrates details of a patient monitoring system 100 in a schematic form. Typically a sensor 106 includes energy emitters 216 located on one side of a patient monitoring site 218 and one or more detectors 220 located generally opposite. The patient monitoring site 218 is usually a patient's finger (as pictured), toe, ear lobe, or the like. Energy emitters 216, such as LEDs, emit particular wavelengths of energy, typically red and infrared light signals, through the flesh of a patient at the monitoring site 218, which attenuates the energy. The detector(s) 220 then detect the attenuated energy and send representative signals to the patient monitor 102 for processing. The patient monitor 102 includes processing board 222 and a host instrument 223. The processing board 222 includes a sensor interface 224, signal processor(s) 226, and an instrument manager 228.

The host instrument typically includes one or more displays 108, control buttons 110, a speaker 112 for audio messages, and a wireless signal broadcaster 234. Control buttons 110 may comprise a keypad, a full keyboard, a track wheel, and the like. A patient monitor 102 can include buttons, switches, toggles, check boxes, and the like implemented in software and actuated by a mouse, trackball, touch screen, or other input device.

The sensor interface 224 receives the signals from the sensor 106 detector(s) 220 and passes the signals to the processor(s) 226 for processing into representations of physiological parameters. These are then passed to the instrument manager 228, which may further process the parameters for display by the host instrument 223. The processor(s) 226 may also communicate with a memory 230 located on the sensor 106; such memory typically contains information related to the properties of the sensor that may be useful in processing the signals, such as, for example, emitter 216 energy wavelengths. The elements of processing board 222 provide processing of the sensor 106 signals. Tracking medical signals is difficult because the signals may include various anomalies that do not reflect an actual changing patient parameter. Strictly displaying raw signals or even translations of raw signals could lead to inaccurate readings or unwarranted alarm states. The processing board 222 processing generally helps to detect truly changing conditions from limited duration anomalies. The host instrument 223 then is able to display one or more physiological parameters according to instructions from the instrument manager 228, and caregivers can be more confident in the reliability of the readings.

### Physiology Background

When oxygen molecules come into contact with blood, the majority of the oxygen molecules are bound to the hemoglobin in red-blood cells and a small portion is dissolved directly in the blood plasma. Both of these processes are driven by the partial pressure of oxygen. In the lung, oxygen diffuses across the alveolar membrane, and then the red cell membrane in lung capillaries. When an oxygen molecule encounters a molecule of hemoglobin, it wedges itself between the iron atom and a nitrogen atom attached to the globin chain. This helps to hold the heme group in place in the protein. One molecule of hemoglobin with its four heme groups is capable of binding four molecules of diatomic oxygen, O₂. The pigment of the oxygen loaded heme group, which is called oxyhemoglobin, is a brilliant red color. This is typically the color of arterial blood. Pressure from dissolved oxygen in plasma and in the surroundings in the red cell helps to keep the oxygen on its binding site.

As the blood circulates to the periphery, the small amount of plasma dissolved oxygen is consumed first by cells in organs and tissues, which causes a drop in the partial pressure of oxygen. This release in pressure makes available the much larger reservoir of heme-bound oxygen which begins a sequential unloading of its four oxygen molecules. At the most, under normal circumstances only 3 molecules of oxygen are unloaded. Partially or fully unloaded hemoglobin is called deoxyhemoglobin. It is a dark blue to purplish color. This is also the typical color of venous blood.

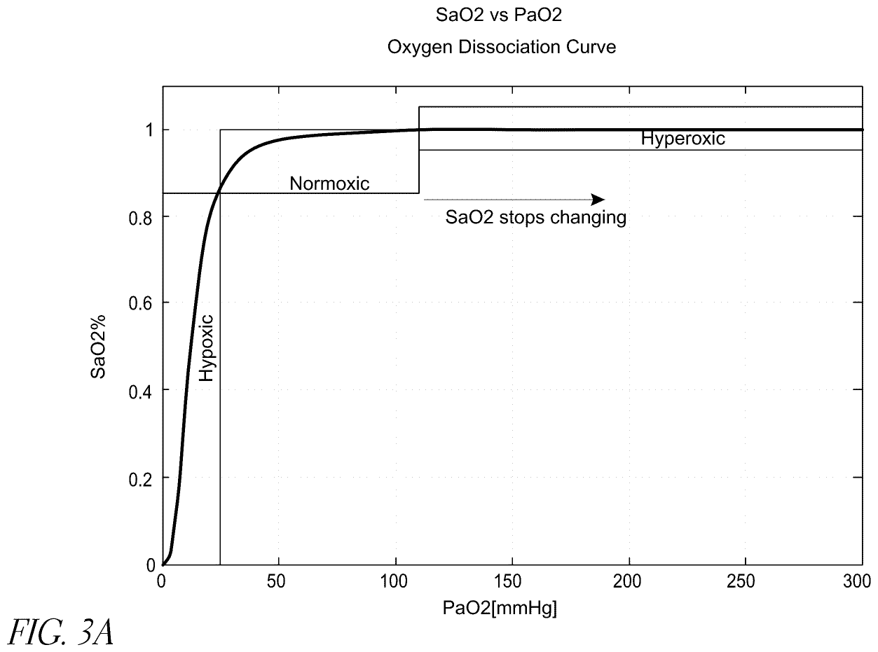

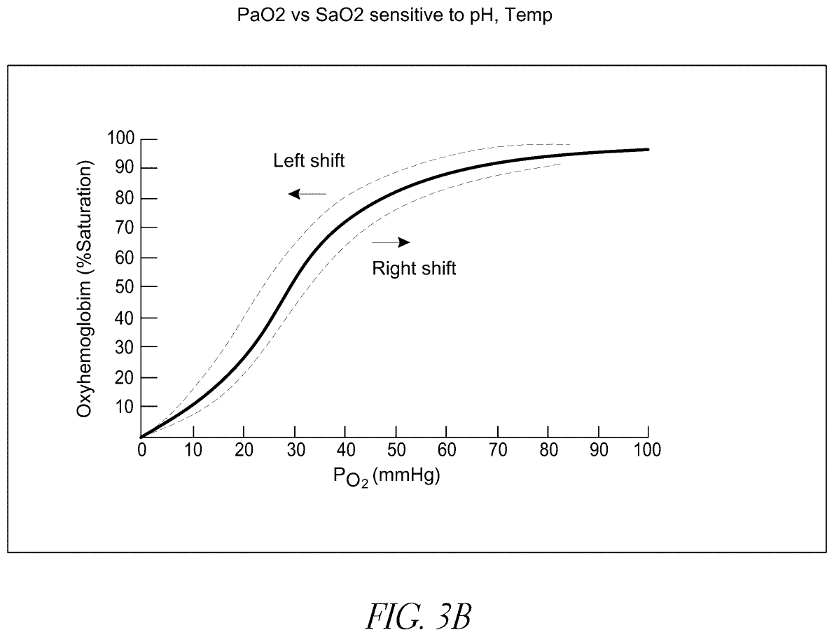

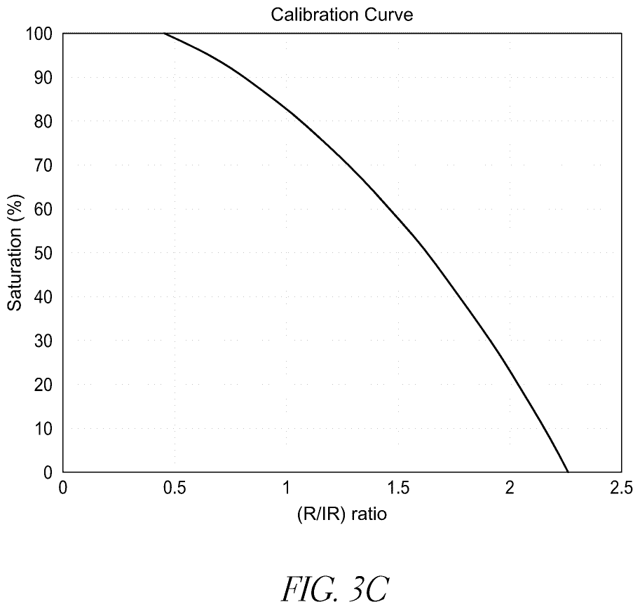

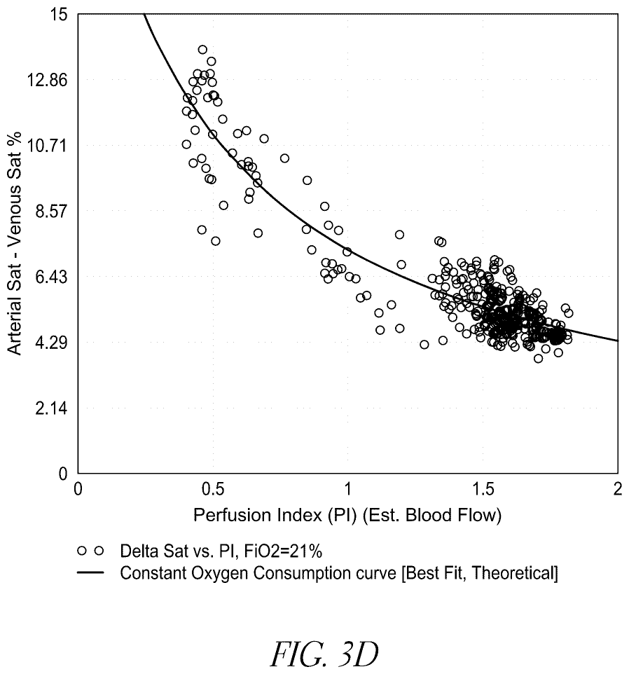

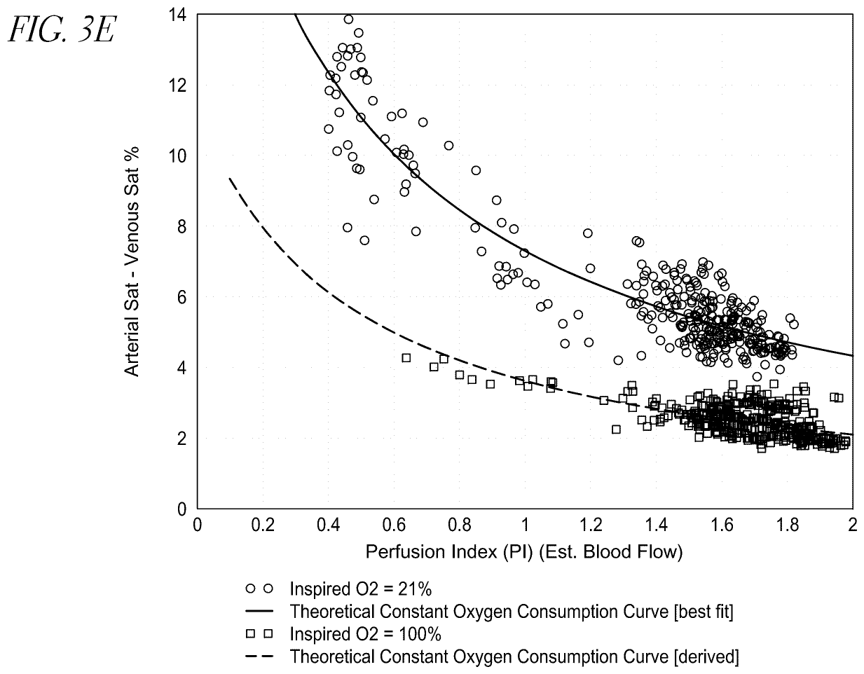

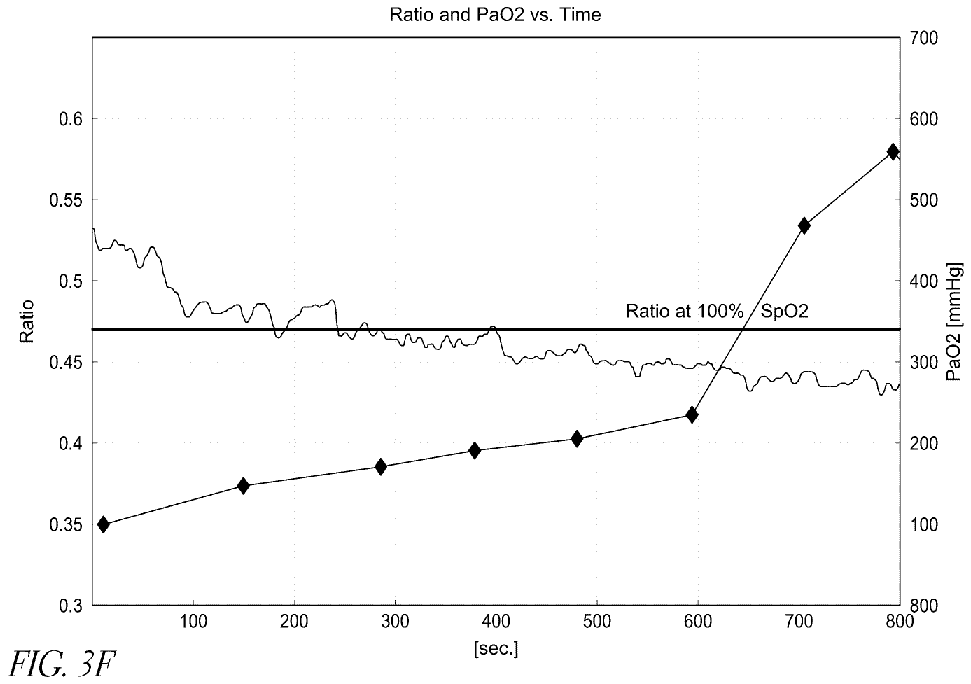

There is a general relationship between the oxygen saturation in blood and the partial pressure of oxygen. This nonlinear relation is described by the oxygen dissociation curve as shown in FIG. 3A. FIG. 3A illustrates a graph of SaO₂ versus the partial pressure of oxygen dissolved in the arterial blood, PaO₂. As the partial pressure of oxygen in the arterial blood increases, the percentage of oxygen saturation of the hemoglobin will increase. After the SaO₂ level reaches 100%, the PaO₂ level continues to rise, but the SaO₂ level will not rise further. Thus, although it is possible to estimate PaO2 levels when SaO2 is below 100%, as illustrated in FIG. 3A, after a certain point, very large changes in the PaO₂ will produce little change in the SaO₂. A patient whose physiology falls on the first part of the curve is commonly referred to as the Hypoxic. As can be seen from FIG. 3A, there is a high sensitivity around PaO₂=30 mmHg, i.e. the slope is large. A patient whose physiology falls on the second part of the curve where SaO₂ begins to level off is Normoxic. In the last portion of the curve, where SaO₂ has reached 100%, a patient is considered Hyperoxic.

FIG. 3B illustrates a graph showing the potential shift in the disassociation curve based on an individual patients response. For example a left shift may occur with decreased temperature, decreased 1,3-diphosphoglycerate (2,30DPG), increased pH, or higher CO in the blood. As another example, a right shift will occur with reduced affinity, increased temperature, increased 2,3-DPG and decreased pH. Thus, there is some-uncertainty when determining PaO₂ based on the SaO₂ measurement. This uncertainty can be reduced if the pH and temperature are given as inputs to the device where an appropriate curve may be selected.

### Oxygen Consumption

The following oxygen content equation relates the amount of oxygen present in the blood given certain hemoglobin concentration (tHb) and partial pressure of oxygen (PaO₂)

$${ContO}_{2} ( {O}_{2} mL / dL ) = tHb ( gramHb / dL ) \cdot 1.34 ( ml {O}_{2} / gramHb ) \cdot {SaO}_{2} + 0.0031 ( ml O 2 / mmHg / dL ) \cdot {PaO}_{2} ( mmHg ) \qquad Eq . 1$$

Alternatively, the Oxygen Content can be measured directly using a Masimo Rainbow Pulse Oximeter available from Masimo Corporation of Irvine, Ca.

Tissues need a requisite amount of O2 molecules for metabolism. Under steady state conditions the O2 consumption is fairly constant. In order to quantify the relationship between oxygen transport and its consumption the Fick principle can be applied. The essence of the Fick principle is that blood flow to an organ can be calculated using a marker substance if the following information is known:
Amount of marker substance taken up by the organ per unit time
Concentration of marker substance in arterial blood supplying the organ
Concentration of marker substance in venous blood leaving the organ

In Fick's original method, the “organ” was the entire human body and the marker substance was oxygen.

This principle may be applied in different ways. For example, if the blood flow to an organ is known, together with the arterial and venous concentrations of the marker substance, the uptake of marker substance by the organ may then be calculated.

As discussed above, hemoglobin and plasma are the main oxygen vectors in the blood. The oxygen content equation can be combined with the Fick principle to describe oxygen consumption and its relationship to blood flow as shown below in Eq. 2.

$$OC = Ca \cdot [ 1.34 \cdot tHb \cdot ( SaO 2 - SvO 2 ) + 0.0031 \cdot ( PaO 2 - PvO 2 ) ] \qquad Eq . 2$$

Where OC is Oxygen consumption (mL/min), Ca is Cardiac output (i.e., local blood flow at the test site (dL/min)), tHb is the Total hemoglobin (gram/dL), SaO2 is Arterial saturation fraction (0-1.0), SvO2 is Venous saturation fraction (0-1.0), PaO2 is the Partial pressure of oxygen in the arterial blood (mmHg), PvO2 is the Partial pressure of oxygen in the venous blood (mmHg), 1.34 represents the HbO2 carrying capacity (mL O2/gram Hb), and 0.0031 represents O2 solubility coefficient in blood (mL O2/dL).

### Noninvasive Oxygen Saturation Measurement

Pulse oximetry was invented by Dr. Ayogi in the 1972 as a technique to measure arterial oxygen saturation noninvasively. Dr. Ayogi was able to isolate the arterial pulse absorption from tissue, bone and cartilage absorptions by looking at a signal synchronous with the heartbeat reflecting the local blood flow at the measurement site. This signal is called the photo-plethysmograph and it can be isolated by the use of a high-pass filter. By exploiting the predictable relationship between arterial oxygen saturation and light absorption through a vascular bed, the arterial blood oxygen saturation (SpaO₂) can be calculated noninvasively. Note that the addition of a small p to SaO₂ to denote calculation from an arterial pulse. It can be shown that the use of two distinct light sources, Red (R)=660 nm and Infrared (IR)=910 nm, a pulse oximeter can calculate the oxygen saturation noninvasively by relating a ratio=R (AC/DC)/IR (AC/DC) to the hemoglobin oxygen saturation through a typical pulse oximeter calibration curve shown in FIG. 3C. We will refer to this ratio as (R/IR) ratio.

Modifying Eq. 2, if (SaO₂−SvO₂) is replaced with ΔSat, (PaO₂−PvO₂) replaced with ΔP, Ca replaced with the local blood flow (BF), the oxygen consumption is set to a constant and the equation is solved for BF, Eq. 3 results:

$$BF = Const / [ 1.34 \cdot tHb \cdot Δ Sat + 0.0031 \cdot Δ P ] \qquad Eq . 3$$

Eq. 3 shows an inverse relationship between blood flow and the arterio-venous saturation difference, ΔSat, as well as arterio-venous O₂ partial pressure difference (ΔP). At normal inspired oxygen levels, the majority of the oxygen is supplied by the hemoglobin. But when the concentration of inspired oxygen is raised, its partial pressure increases, hence ΔP, and more oxygen is delivered to the tissue through the O₂ dissolved in the plasma. Based on Eq. 3, if we consider a digit where a pulse oximeter probe is placed, the increase of inspired oxygen partial pressure will lead to a decrease in the arterio-venous ΔSat. This is true whenever the oxygen consumption is relatively constant.

In a vascular bed the arterial vasculature is coupled mechanically to the venous vasculature through the tissues. Although this coupling is small, the optical arterial pulse, e.g. photo-plethysmograph, has invariably a small venous component. This component is not fixed across subjects but its average is indirectly calibrated for in the saturation calibration curve. Its effect on the arterial pulse is proportional to the coupling size as well as the difference between the arterial and venous saturations at the site. Let us consider a typical subject at room-air saturation of 98%. Looking at the saturation calibration curve of FIG. 3C, a (R/IR) ratio of 0.53 corresponds to 98% saturation. If the inspired oxygen concentration is increased beyond the normal O2=21%, the (R/IR) ratio continues to drop below 0.53. An example is shown in FIG. 3F where the ratio starts at 0.43 and goes down to 0.43. It can even reach a level as low as 0.3 on some subjects at an inspired O2=100%.

This behavior may be explained by the reduction in the optical effect of venous coupling as the delta saturation between the arterial and the venous is reduced due to the increase in availability of plasma oxygen. Under this condition, the venous blood will look, optically, a lot like the arterial blood. Hence, the size of the Red photo-plethysmograph signal will shrink with respect to the IR indicating a shrinking ΔSat, i.e. higher venous saturation. In 1995, Masimo Corporation (Masimo) introduced a new technique for calculation the venous oxygen saturation (SpvO₂) by introducing an artificial pulse into the digit (see, e.g., U.S. Pat. No. 5,638,816, incorporated herein by reference). By using a pulse oximeter with a probe and a subject's digit, a continuous measure of SpaO₂ and SpvO₂ can be calculated. The blood perfusion index (PI) is used as a proxy for the blood flow to the digit. FIG. 3D depicts such an inverse relationship between blood flow (BF) and arterio-venous saturation ΔSat.

FIG. 3E depicts the effect of increasing the inspired O₂ concentration on the calculated ΔSat. As expected there is a commensurate reduction in the ΔSat with the increase of oxygen concentration. The arterio-venous ΔSat will continue to decrease if the oxygen pressure is increased beyond atmospheric pressure. However, a point of diminishing return will be reached where no more change is possible. At that point the R/IR ratio will stop changing as shown in FIG. 3F. The increase in PaO₂ can be indirectly monitored beyond the normal 100 mmHg by looking at the effects of shrinking ΔSat. This cannot be done by looking at the SaO₂ as it will plateau at 100%.

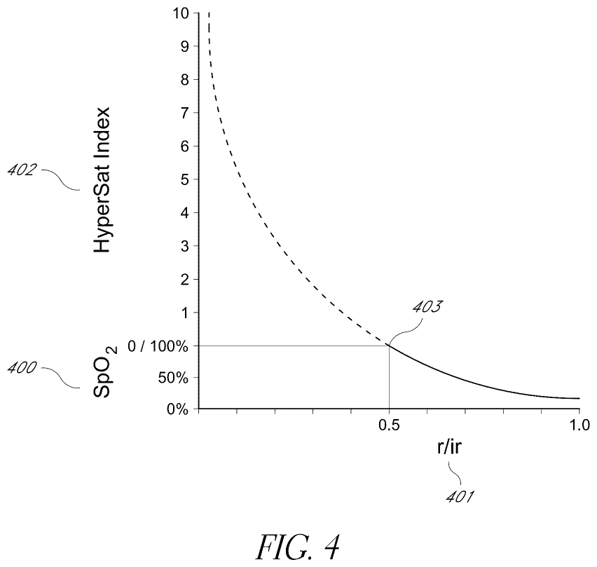

FIG. 4 illustrates a graph of SpO₂ saturation percentage 400 versus the R/IR ratio 401 according to an embodiment of the invention. In the illustrated example, the R/IR ratio is at 0.5 when the SpO₂ maxes out at 100%. While the SpO₂ level will max out at 100% saturation, the R/IR ratio continues to drop when more oxygen is dissolved in the blood. An embodiment of the invention calls for calculating a hypersaturation index 402 based on the R/IR ratio after the point 403 where the R/IR ratio translates to a SpO₂ level of 100% saturation. This hypersaturation index 402 assists medical practitioners in exercising their judgment in ensuring that the patient's blood is not too oversaturated with oxygen. In another embodiment, the hypersaturation index is calculated in response to a user signal, i.e., not necessarily at the point where the SpO₂ level is at 100% saturation.

Determining a level of hypersaturation is particularly important in a variety of patient types. For example, patients on supplemental O₂ titration are at risk of complications caused by hypersaturation. Patients on a ventilator or where FiO2 therapy is given to the patient are also at risk. Further, closed loop positive pressure O₂ delivery or FiO₂ delivery devices also place a patient at risk of hypersaturation. This may include, for example, CPAP machines or those suffering obstructive sleep apnea.

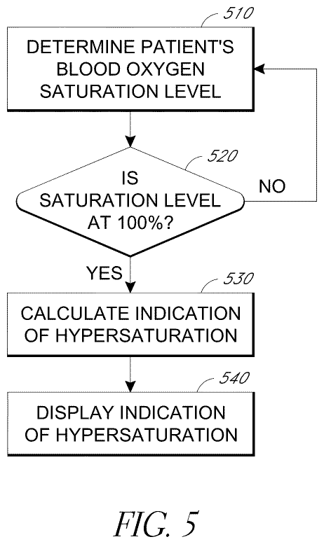

In an embodiment of the invention, the patient's oxygen saturation level SpO₂ is determined and monitored. When the saturation level reaches 100%, an indication of rising oxygen levels, such as a hypersaturation index, is calculated. The indication of rising oxygen levels may also be displayed on an output device such as the display 108 in FIG. 1. FIG. 5 is a flowchart that illustrates this embodiment of the invention. In this embodiment, the patient's blood oxygen saturation level SpO₂ is determined at step 500. If the blood oxygen saturation level maxes out at 100% at step 520, an indication of hypersaturation is calculated at step 530 and displayed at step 540.

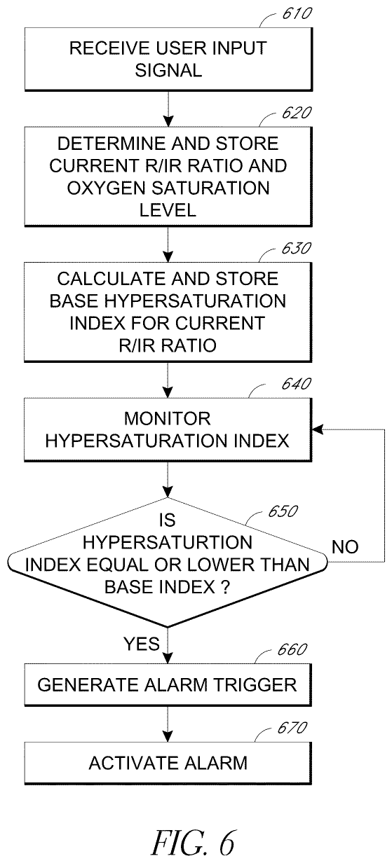

In another embodiment of the invention, illustrated in FIG. 6, the patient's oxygen saturation level SpO₂ is determined and stored at step 620 in response to a signal from the user at step 610. The signal typically indicates that a medical procedure is about to begin. A base hypersaturation index value is then calculated at step 630 based on the stored oxygen saturation level and the R/IR ratio. The hypersaturation index is then monitored at step 640 as the patient's oxygen saturation level changes. Next, an alarm trigger is generated at step 660 when the hypersaturation index value is less than or equal to the base hypersaturation index value as determined in step 650. Finally, an alarm is activated at step 670 in response to the alarm trigger.

In an alternative embodiment, the oximeter monitors a patient and automatically determines a baseline oxygen saturation level and/or baseline ratio from stable measurements taken when the oximeter first begins measurements. The oximeter can indicate that a baseline measurement has been determined or can indicate that it is unable to determine a baseline measurement if stable measurements cannot be obtained. Once a baseline measurement is obtained, the oximeter will monitor the patient for an inflection point in the saturation and ratio calculations. If the oximeter finds an inflection point where the patient's oxygen saturation begins to rise and/or ratios begin to fall, it will determine that oxygen is being administered to the patient. In this way, a caregiver is not required to push a button or otherwise indicate the start of a procedure or the start oxygen administration. Along the same lines, once a patient is hypersaturated, the oximeter will monitor the saturation level and/or ratio calculations of the patient for an inflection point indicating that oxygen is no longer being administered to the patient. Again the oximeter will alarm when the oxygen saturation values and/or ratios return to their normal baseline levels.

In yet another embodiment of the invention, a maximum hypersaturation index value is also calculated and stored in response to a user signal. In this embodiment, an alarm trigger is generated when the monitored hypersaturation index value is more than or equal to the maximum hypersaturation index value.

In an alternative embodiment, a visual oxygen hypersaturation alarm is activated. The oxygen hypersaturation alarm may include text that indicates that the oxygen hypersaturation index has dropped below the base hypersaturation index value. In another embodiment, the alarm may include text that indicates that the oxygen hypersaturation index has exceeded a threshold value. The visual oxygen hypersaturation alarm may be accompanied or replaced by an audio alarm in certain embodiments.

FIG. 7 illustrates an example of a visualization of an indication of hypersaturation according to an embodiment of the invention. This visualization can be displayed on a display, such as the display 108 in FIG. 1. In the illustrated embodiment, the indicator is displayed as a speedometer-type visualization. The display includes a pointer 700 that points to the current value of the hypersaturation indicator. The value, for example, can be on a scale of 0-100 or 0-10 to differentiate from oxygen saturation. In one embodiment, the spectrum of possible levels may be indicated by various shades or colors. For example, the low range of values may be indicated by an area 701 that is green in color, the medium range in values may be indicated by an area 702 that is orange in color, and the high range in values may be indicated by an area 703 that is red in color.

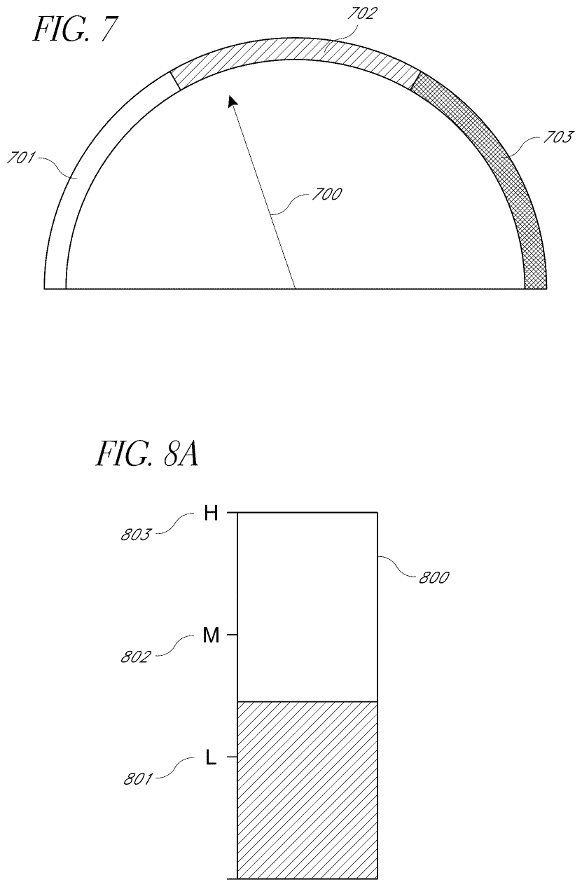

FIG. 8A illustrates another example of a visualization of an indication of hypersaturation according to an embodiment of the invention. This visualization can also be displayed on a display, such as the display 108 in FIG. 1. In the illustrated embodiment, the hypersaturation indicator is displayed as a bar 800. In one embodiment, the size of the area of the bar that is shaded or colored depends on the value of the hypersaturation indicator. For example, a low value may be represented by a small shaded area below the “L” level 801. A medium value may be represented by a larger shaded area that remains below the “M” level 802. Finally, a high value may be represented by an even larger shaded area that can cover the entirety of the bar up to the “H” level 803.

FIG. 8B illustrates yet another example of a visualization of an indication of hypersaturation. The displayed graph 820 illustrates hypersaturation on a scale of 0-100%. The line 821 illustrates an estimated hypersaturation value. The shaded area 823 illustrates the variability of the hypersaturation index. In other words, each patient's physiology is different and depending patient, their hypersaturation my not exactly follow the population average. This is explained in more detail, for example, with respect to FIG. 3B. Thus, the shaded area 823 provides an indication of the uncertainty in the estimate 821. This provides a care provider with a better indication of the actual hypersaturation that the patient is experiencing. In the embodiment of FIG. 8B, 0% represents no detectable oxygen reserve, or no indication of hypersaturation. 100% indicates a maximum detectable reserve or a maximum hypersaturation.

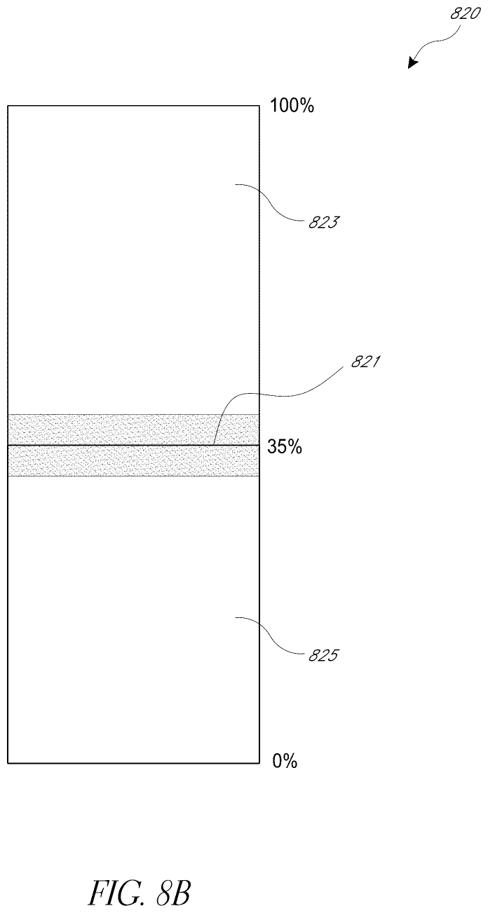

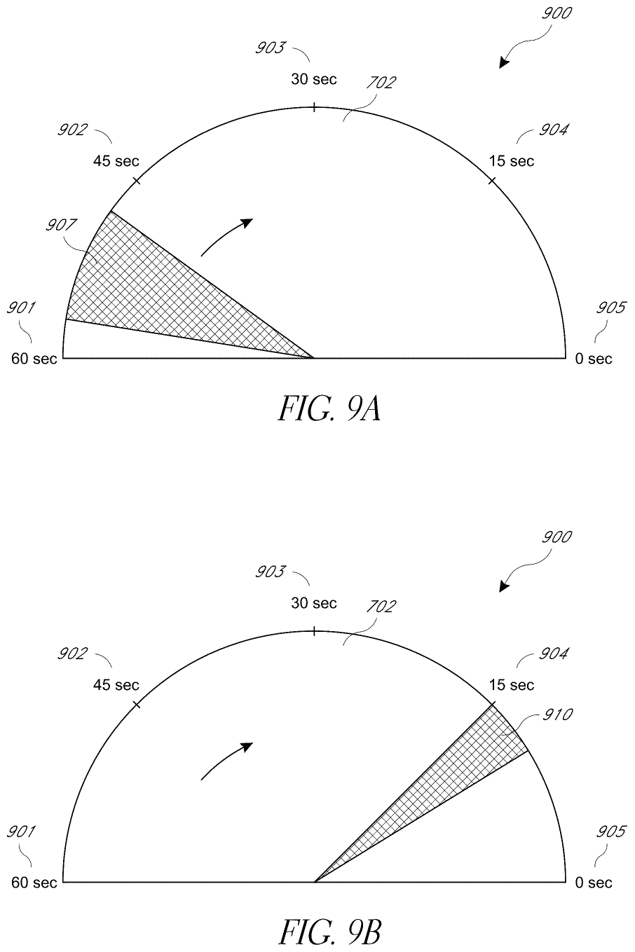

FIG. 9A illustrates an embodiment of a hypersaturation timer 900. A hypersaturation timer 900 is useful, for example, during procedures such as a patient intubation when the patient is forced to stop breathing. The timer provides an indication of the amount of time a care giver has before the patient returns from a hypersaturated state to a baseline saturation state. The timer includes a countdown indications 901-905. In the embodiment of FIG. 9A, the countdown begins at about 60 seconds and counts down to zero. When the counter is initially started, the amount of time a patient will take to return to a baseline saturation state is relatively difficult to determine. Thus, the timer 900 provides a range of time left which is illustrated by shaded area 907. The shaded area moves clockwise around the timer indicating a range of time left before the patient reaches a baseline state. As time goes by, the amount of time a patient will take to return to a baseline saturation state becomes more predictable based on how quickly the ratios change. Thus, as illustrated in FIG. 9B, the range indicated by the shaded area 910 becomes smaller.

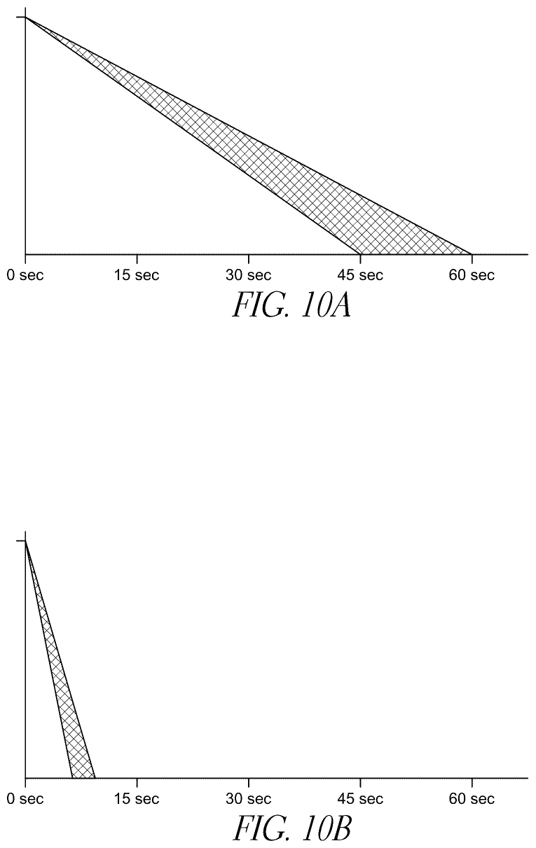

FIG. 10 illustrates another embodiment of a timer 1000. Similar to FIGS. 9A and B, timer 1000 has a count-down range 1002 that decreases as time expires and the time in which a patient returns to their base line saturation becomes more certain.

In another embodiment not shown, a simple digital count-down clock could also be used to indicate the amount of time left for a hypersaturation patient to return to their baseline saturation level. The count-down clock can indicate a range or it can simple indicate a number and speed up or slow down based on the rate of return experienced by the patient.

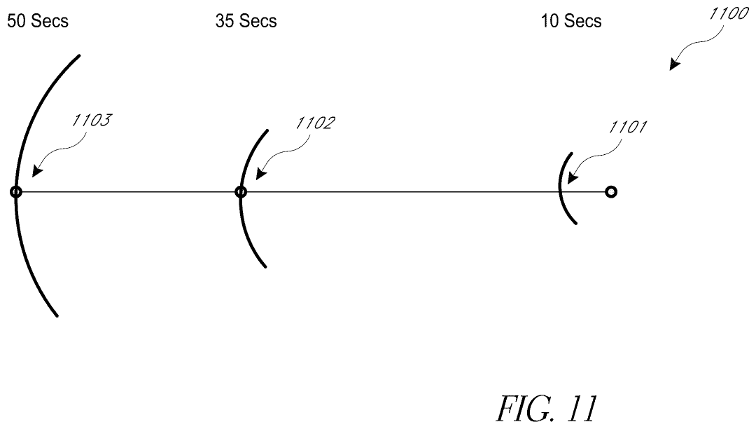

FIG. 11 illustrates an embodiment count down display of an oxygen reserve, or the time left for a hypersaturation patient to return to baseline saturation. Put in other terms, the time in seconds starts increasing from zero as a subject transitions from normoxia to hyperoxia. The display then decreases when the subject transitions from the Hyperoxic state to the Normoxic state. The display of FIG. 11 includes an arc indicator, for example, arc indicators 1101, 1102, and 1103. The indicators are arced in order to show the uncertainty range in the time left in the display. Although the arcs 1101, 1102, and 1103 are all illustrated on the display for illustration and explanation purposes, it is to be understood that during measurement, only a single arc is displayed which according to the relative time

Although the foregoing has been described in terms of certain specific embodiments, other embodiments will be apparent to those of ordinary skill in the art from the disclosure herein. Moreover, the described embodiments have been presented by way of example only, and are not intended to limit the scope of the disclosure. Indeed, the novel methods and systems described herein may be embodied in a variety of other forms without departing from the spirit thereof. Accordingly, other combinations, omissions, substitutions, and modifications will be apparent to the skilled artisan in view of the disclosure herein. Thus, the present disclosure is not limited by the disclosed embodiments, but is defined by reference to the appended claims. The accompanying claims and their equivalents are intended to cover forms or modifications as would fall within the scope and spirit of the disclosure.

1. (canceled)

2. A method of monitoring a hypersaturation index of a patient using a pulse oximeter device, the method comprising:
emitting light of at least two wavelengths from one or more light emitters of the pulse oximeter device;
detecting the emitted light after attenuation by tissue of a patient by a light detector of the pulse oximeter device;
providing a signal representative of the detected emitted light to a signal processor;
processing the signal to determine a ratio of the at least two wavelengths;
calculating an oxygen saturation level of the patient based on the ratio;
calculating a hypersaturation index of the patient based on the ratio prior to the oxygen saturation level reaching 100%, wherein the hypersaturation index is different from the oxygen saturation level; and
displaying the oxygen saturation level and the hypersaturation level prior to the oxygen saturation level reaching 100%.

3. The method of claim 2, wherein the oxygen saturation level is measure of a percentage of available carriers in the patient's blood that are bound to oxygen molecules.

4. The method of claim 3, wherein the hypersaturation index is responsive to a quantity of oxygen dissolved in the patient's blood and not bound to any of said carriers.

5. The method of claim 2, wherein the hypersaturation index is responsive to a quantity of oxygen dissolved in the patient's blood and not bound to any carriers.

6. The method of claim 2, wherein the hypersaturation index is responsive to an oxygen reserve of the patient.

7. A method of monitoring a hypersaturation index of a patient using a pulse oximeter device, the method comprising:
emitting light of at least two wavelengths from one or more light emitters of the pulse oximeter device;
detecting the emitted light after attenuation by tissue of a patient by a light detector of the pulse oximeter device;
providing a signal representative of the detected emitted light to a signal processor;
processing the signal to determine a ratio of the at least two wavelengths;
calculating an oxygen saturation level of the patient based on the ratio at a first timepoint, wherein the patient is in a normoxic state at the first timepoint;
calculating a base hypersaturation index based on the ratio at the first timepoint;
monitoring a hypersaturation index as the patient's ratio and the oxygen saturation level changes after the first timepoint, wherein the oxygen saturation level is at or below 100%; and
outputting an alarm when the hypersaturation index falls below the base hypersaturation index.

8. The method of claim 7, wherein the ratio corresponds to a hypoxic state of the patient when the hypersaturation index falls below the base hypersaturation index.

9. The method of claim 7, wherein the patient is intubated and the first timepoint corresponds to a start of a medical procedure.

10. The method of claim 9, wherein the base hypersaturation index is calculated in response to a user signal.

11. The method of claim 9, wherein the base hypersaturation index is automatically calculated in response to stable measurements of the oxygen saturation level.

12. The method of claim 9, further comprising determining an inflection point in values of the ratio indicating that oxygen is being administered to the patient.

13. The method of claim 12, further comprising outputting a hypersaturation alarm after the inflection point indicating that oxygen is being administered to the patient, in response to the hypersaturation index exceeding a maximum value.

14. The method of claim 9, further comprising determining an inflection point in values of the ratio indicating that oxygen is no longer being administered to the patient.

15. The method of claim 14, wherein the alarm is outputted after the inflection point indicating that oxygen is no longer being administered to the patient.

16. The method of claim 7, further comprising displaying the oxygen saturation level and the hypersaturation level prior to the oxygen saturation level reaching 100%.

17. The method of claim 7, wherein the oxygen saturation level is measure of a percentage of available carriers in the patient's blood that are bound to oxygen molecules.

18. The method of claim 17, wherein the hypersaturation index is responsive to a quantity of oxygen dissolved in the patient's blood and not bound to any of said carriers.

19. The method of claim 7, wherein the hypersaturation index is responsive to a quantity of oxygen dissolved in the patient's blood and not bound to any carriers.
